SEQUENCE LISTING (2 sequences)

SEQ ID NO: 1 - protein, 5 aa, artificial sequence
  synthetic peptide
  MOD_RES (1)..(1): amidation by a Palmitoyl chain on the N terminal end
KTTKS

SEQ ID NO: 2 - protein, 5 aa, artificial sequence
  synthetic peptide
  MOD_RES (1)..(1): amidation by a Palmitoyl chain on the N terminal end
KTSKS

(19) United States
(12) Patent Application Publication
(10) Pub. No.: US 2025/0302720 A1
(43) Pub. Date: Oct. 2, 2025

(54) PEPTIDE-BASED HAIR TREATMENT

(71) Applicant: Sederma, Le Perray-en-Yvelines (FR)

(72) Inventors: Philippe Mondon, Montrouge (FR); Caroline Ringenbach, Rambouillet (FR); Céline Bondil, Rambouillet (FR); Nathalie Rouyer, Plaisir (FR)

(73) Assignee: Sederma, Le Perray-en-Yvelines (FR)

(21) Appl. No.: 18/249,338

(22) PCT Filed: Oct. 18, 2021
(86) PCT No.: PCT/EP2021/078819

§ 371 Date: Apr. 17, 2023

(30) Foreign Application Priority Data
Oct. 21, 2020 (FR) 2010784

(51) Int. Cl. A61K 8/64; A61Q 5/00

(52) U.S. Cl. CPC A61K 8/64; A61Q 5/004

(57) ABSTRACT

The treatment is cosmetic non-therapeutic, adapted to prevent hair depigmentation and implementing a peptide X-(Xaa)n-P*P*-(Xaa)m-Z, wherein P* corresponds to a Proline, analog or derivative thereof, Xaa is an amino-acid selected from P*, Glycine, Alanine, Valine, Leucine, Isoleucine, Arginine and Phenylalanine, n and m are equal to 0, 1, 2, 3 or 4, with n+m≤8; and at the N-terminal end, X is selected from H, —CO—R¹, —SO₂—R¹ or a biotinoyle group; at the C-terminal end, Z is selected from OH, OR¹, NH₂, NHR¹ or NR¹R²; R¹ and R² being selected from an alkyl, aryl, aralkyl, alkylaryl, alkoxy, saccharide and aryloxy groups having 1 to 24 carbon atoms. The preferred peptide is the Pal-PP used in an anti-greying treatment.

### TECHNICAL FIELD

The present invention relates to a peptide-based hair treatment, including body hairs and head hair (including scalp hair, beard hairs, eyelashes, and eyebrows), of mammals, animals, or humans. It relates more particularly to a treatment of canities (from greying to whitening), namely a treatment linked to hair depigmentation. It concerns the cosmetics, hygiene and personal care products and dermo-pharmacy industries.

### BACKGROUND ART

The hair life takes place in a so-called pilar cycle, according to three phases that are repeated until the hair dies. Thus, the hair does not grow continuously, but in successive cycles. The genetically programmed number of hair cycles is twenty-five to thirty in total, for a lifetime.

The hair cycle begins with a phase called anagen, which is when the hair grows strongly inside its hair follicle. It lasts an average of two to five years. 85-90% of the hair is usually in the anagen phase. During this phase, the hair bulb located at the lower end of the follicle regenerates and then produces the hair fiber. The hair thus grows about one cm per month. It is a highly proliferative phase which results from the proximity between the dermal papilla, in contact with the blood vessels, and the swollen part of the sheathn, called the hair bulge, where the hair stem cells (of melanocyte and keratinocyte types) are found. The origin of hair fiber producing will occur in the hair bulb thanks to the intense production of keratins and keratinocytes that will form the external cuticles of the hair and the cortex.

At the end of this anagen phase, the follicle enters a regression phase called the catagen phase and the synthesis are stopped. The fiber no longer grows due to the lack of nourishment of the dermal papilla from the blood vessels. This phase lasts only a few weeks and affects around 2 to 3% of the hair at a given time. The follicle retracts and gradually involutes. The keratinocyte-type cells and the melanocytes progressively disappear and the bulb collapses on itself while retaining the dermal papilla.

Then begins the rise of the dermal papilla towards the bulge. This is the phase called telogen that precedes a new anagen phase. 8-10% of the hairs on a scalp are in the telogen phase at a given time. They stay in this phase for about 6 to 7 months for a young person, but longer as age increases.

This hair cycle is the same for the body hairs with different phase durations. In particular the duration of the anagen phase is shorter for the body hairs.

The natural coloring of hair is linked to the existence of two types of melanin: eumelanin, a brown-black pigment, and pheomelanin, a red-yellow pigment. The balance between these two pigments conditions all the shades of hair observed. However, like all organs, hair is ageing, which results in less pigmentation.

The starting point for melanin synthesis is L-Tyrosine, which is the initial substrate of tyrosinase (TYR) whose activity produces dopaquinone, a molecule that is the source of both types of melanin. Eumelanin is synthesized through several transformations with the intervention of other enzymes such as TYRP1 and TYRP2. Phaeomelanin takes another route and uses the sulfur amino acid L-cysteine. Certain proteins also have a determining role in this synthesis. In addition to the three enzymes mentioned above, the MITF protein triggers the production of TYR and TYRP1, and therefore of melanin. There is also the CREB protein which is used to trigger the production of MITF.

Melanin production in the hair follicle is cyclical and occurs only in the anagen phase. This production takes place in the melanocytes. The melanin is stored in melanosomes and these melanosomes are then distributed to surrounding keratinocytes thanks to dendrites (kinds of tentacles, extensions of melanocytes). Thereafter, the keratinocytes phagocyte the melanosomes, and the more active the phagocytosis is, the better the pigmentation of the hair is.

The greying or the canitie is very age related. A genetic factor is also often mentioned in the acceleration of canitie precocity, as well as environmental factors in the broadest sense.

In grey hairs, there is a gradual decrease in the pigmentation of the fiber. A certain consensus exists around the fact that the greying is linked to a more or less strong reduction of the active melanocytes in the bulb of grey hair under the effect of age and oxidative stress. This leads to less production of melanin and melanosomes as well as a reduction in the dendrite size and a decrease in the transfer of melanosomes to keratinocytes. There is also less tyrosinase activity and the melanocyte shows greater cytoplasm vacuolation, indicating oxidative stress. In white hair, there are no melanocytes in the bulb, the latter being in fact unable to migrate during a new hair cycle. Melanogenesis, which is very active in hairs throughout life, produces many radical and oxidizing species including H₂O₂ (hydrogen peroxide), which over several months and years causes cumulative oxidative damages to the melanocyte. Thus, grey hair follicles are significantly associated with more mitochondrial DNA damages and more apoptosis. The BCL-2 protein has an antioxidant role and protects the melanocyte against death by apoptosis. It limits the greying.

With age, the antioxidant defenses of the cell are reduced, in particular the catalase production responsible for the detoxification of H₂O₂ into oxygen and water is reduced, as well as gluthathione production. This has cytotoxic implications for the hair follicle and accelerates the greying.

To remedy the greying, women often use artificial hair dyes that hold well over time, but often use chemical fixatives, that can be sensitizing. This process, however, is not in the culture of men.

Therefore, there is a real need for a biologically active compound which both delays the depigmentation of hair, and also promotes their repigmentation, in particular to avoid or delay the need to use artificial colorings.

### SUMMARY OF THE INVENTION

To this aim, the applicant provides the use of at least one peptide of Formula 1:

X-(Xaa)n-P*P*-(Xaa)m-Z

wherein:
P* corresponds to a Proline, analog or derivative thereof;
Xaa is an amino-acid selected from P*, Glycine, Alanine, Valine, Leucine, Isoleucine, Arginine and Phenylalanine, analogs and derivatives, the Xaa being selected independently from each other;
n and m are integers selected indepently from each other equal to 0, 1, 2, 3 or 4, with n+m≤8; and
at the N-terminal end, X is selected from H, —CO—R¹, —SO₂—R¹ or a biotinoyle group;
at the C-terminal end, Z is selected from OH, OR¹, NH₂, NHR¹ or NR¹R²;
R¹ and R² are, independently from each other, selected from an alkyl, aryl, aralkyl, alkylaryl, alkoxy, saccharide and aryloxy groups, that can be linear or branched, cyclic, polycyclic, unsaturated, hydroxylated, carbonylated, phosphorylated and/or sulfured, said group having 1 to 24 carbon atom and optionally having in its skeleton one or more O, S and/or N heteroatoms; for a cosmetic non-therapeutical hair treatment adapted to prevent hair depigmentation.

The detailed description of in-vitro and in-vivo tests given below shows that the peptide according to the invention offers a long-term action, characterized by an anti-aging effect, aimed at improving the general functioning of the hair system, in particular at the level of the hair follicle, in order to strengthen it and keep it in working order, to prevent it from degrading, so that it continues to produce melanin, to transfer it optimally, and thus to prevent hair depigmentation and the appearance of grey hairs.

The peptide according to the invention exhibits an antioxidant effect on the melanocytes present in the hair follicle and/or promotes the transfer of melanin to the keratinocytes by the dentrites.

The tests also show a complementary and advantageous action on pigmentation stimulation, in-vitro, but above all in-vivo, with in addition an effect which is prolonged over time in this second case (a long-lasting effect).

The peptide according to the invention is therefore particularly advantageous for responding to the general problem of greying hairs by significantly and durably improving this condition.

This dual level of action, both preventive (preventing depigmentation, in particular by postponing it) and curative (re-pigmentation) is particularly advantageous.

In Formula 1, according to the invention, P* corresponds to a Proline, a derivative or an analog thereof. The present invention thus covers the following derivatives or analogs of Proline:
1) from rings of different size (for example 4 or 6 bonds);
2) resulting from the modification of the relative position (α or β) of the acid function (COOH) with respect to the nitrogen of the cycle;
3) resulting from substitutions by a group R on the cycle modifying its size and/or its polarity;
4) resulting from the combination of the previous items.

They can be represented by the following general Formula 1:

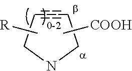

In this Formula 1, the R group can be in any position and the COOH in the α or β position.

The present invention preferably provides the analog compounds presented in the following Table 1, possessing a nitrogen in the cycle such as the Proline (Cycle at 5 and COOH at α):

TABLE 1

| Compound name | Chemical structure |
|---|---|
| azetidine-2-carboxylic acid | 4 atoms cycle and COOH in α |
| β-proline (or pyrrolidine-3- | 5 atoms cycle and COOH in β |
| carboxylic acid) | |
| pipecolic acid | 6 atoms cycle and COOH in α |
| nipecotic acid | 6 atoms cycle and COOH in β |
| thio-proline (or thiazolidine- | 5 atoms cycle and COOH in α + S in |
| 4-carboxylic acid) | position 4 |
| 4-hydroxy-proline | 5 atoms cycle and COOH in α + OH in |
| | position 4 |
| 3,4-dehydro-proline | 5 atoms cycle and COOH in α + |
| | insaturation in 3-4 |

Other compounds resulting from the substitution of proline with a R group are also possible. Non-limiting examples are given in the following Table 2.

TABLE 2

| R group | Position |
|---|---|
| piperidin-4-yl | 1 on nitrogen |
| phenyl; dimethyl | 3 |
| phenyl; OH; NH₂; F; F₂; CF₃; benzyl; cyclohexyl; oxo; | 4 |
| bromobenzyl; SH; phenoxy | |
| phenyl; dimethyl; oxo | 5 |
| phenyl, cyclohexyl | 4 + 5 |

Proline can also be replaced by substituted analogues having a six-link cycle as the examples given in following Table 3:

TABLE 3

| Compound name | Chemical structure |
|---|---|
| 3-carboxy-morpholine | 6 atoms cycle oxygenated + |
| | COOH in position 3 |
| 2-carboxy-morpholine | 6 atoms cycle oxygenated + |
| | COOH in position 2 |
| Tic (or 1,2,3,4-tétrahydroisoquinoline- | 6 atoms cycle oxygenated + |
| 3-carboxylic acid) and its hydroxylated | COOH in position 2 on this |
| derivative | cycle + phenyl in position 4 + 5 |
| Tiq (or 1,2,3,4-tetrahydroisoquinoline- | 6 atom cycle + COOH in |
| 1-carboxylic acid) | position 6 sur on this cycle + |
| | indole in position 4 + 5 |

According to other preferred features of the invention:
the peptide is modified at its N-terminal end and/or C-terminal end (exclusion of the case where X═H and Z═OH) but preferably the peptide is modified only at its N-terminal end. The derivatization of the peptide in the N- and/or C-terminal ends is intended in particular to improve the bioavailability of the peptide, by improving its penetration into the skin. This effect can also be obtained by means of vectorization, for example via encapsulation of the peptide(s).
R¹ and/or R² is an alkyl chain of 1 to 24 carbon atoms, preferably a lipophilic alkyl chain of 3 to 24 carbon atoms; and/or
X is an acyl group CO—R¹ and Z is chosen from OH, OMe, OEt and NH₂, preferably OH; X is preferably chosen from octanoyl (C8), decanoyl (C10), lauroyl (C12), myristoyl (C14), palmitoyl (C16), stearoyl (C18), biotinoyl, elaidoyl, oleoyl and lipoyl; more preferably chosen from a lauroyl (C12), myristoyl (C14) and palmitoyl (C16); and/or
Z is OH and X is chosen from a palmitoyl (C16), myristoyl (C14) and lauroyl (C12); more preferably palmitoyl (C16).

Peptides comprising in the N- or C-terminal end derivatives of specific acids such as those of ascorbic, retinoic, cinnamic, oleanolic, hyaluronic, nicotinic, lipoic, gallic or pantothenic acid are encompassed by the present invention.

As shown on Formula 1, the active peptide sequence P*P* according to the invention can be part of a peptide having additional amino acids Xaa on either side thereof, which are hydrophobic amino acids. Preferably, these Xaa(s) are chosen from P*, Glycine, Alanine, Valine, Leucine, and Isoleucine, more preferably from P*, Glycine and Alanine, more preferably Proline, Glycine and Alanine.

The preferred peptide according to the invention is the X-P*P*-Z peptide, more preferably the X-PP-Z peptide, corresponding to n and m equal to 0.

The particularly preferred peptide according to the invention is Palmitoyl-PP-OH, hereinafter referred to as Pal-PP-OH.

The peptides according to the invention can be optically pure or consist of the L or D isomers or a mixture thereof. L isomers which are those naturally occurring may be preferred.

The present invention also covers derivatives of the amino acids Xaa (with modification and/or addition of a chemical function to one or more of the amino acids but without change in the carbon skeleton) and the analogs (with modification and/or addition of a chemical function to one or more of the amino acids but with in addition a change in the carbon skeleton), and complexes with other species such as a metal ion (eg copper, zinc, manganese, magnesium, and others).

The peptides can be used, where appropriate, in the form of salts, in particular hydrochloric salt, or acetate.

The present invention also provides a composition, in particular topical, comprising at least one peptide according to the invention and a physiologically acceptable medium. Depending on the physiologically acceptable medium and the concentration of peptide(s), this composition may constitute a concentrated active ingredient intended to enter a final composition for a consumer, or may directly constitute said final composition, which is less concentrated in peptide(s).

In a composition according to the invention, the at least one peptide can be present at a higher or lower concentration depending on its destination, ranging from 10-7% to 20% relative to the total weight of the composition, preferably ranging from 10-6% to 10%, more preferably from 10-5% to 5%, by weight relative to the total weight of the composition.

For example, in a composition forming an active ingredient for the treatment according to the invention, the at least one peptide will be present at a high concentration generally ranging from 100 ppm to 20,000 ppm, preferably ranging from 500 ppm to 15,000 ppm, more preferably from 1000 ppm to 10,000 ppm. This ingredient will then be formulated in general between 0.01 and 10%, preferably between 1 and 5% in the final topical preparation.

When several peptides according to the invention are present in a composition according to the invention, they may be present in varying relative proportions, in equivalent amounts, or on the contrary in different proportions.

All the percentages and ratios used in the present application are expressed relative to the weight of the total composition and all the measurements are made at 25° C. unless specified otherwise.

By “physiologically acceptable medium” is meant according to the present invention, without limitation, an aqueous or hydroalcoholic solution, a water-in-oil emulsion, an oil-in-water emulsion, a microemulsion, an aqueous gel, an anhydrous gel, a serum, a vesicle dispersion, or a powder.

“Physiologically acceptable” means that the compositions are suitable for topical or transdermal use, in contact with the mucous membranes, nails, scalp, hair, body hair and skin of mammals and more particularly of humans, compositions which can be ingested or injected into the skin, without risk of toxicity, incompatibility, instability, allergic response, and others. This “physiologically acceptable medium” forms what is conventionally called the excipient of the composition.

The peptide(s) according to the invention can be solubilized in a lipophilic or hydrophilic matrix, optionally with a solubilizer, depending on the intended application.

The peptide(s) in the treatment according to the invention can be combined with other active ingredients at effective concentrations which can act synergistically or in reinforcement to achieve the desired effects described for the invention, such as the following agents: soothing, moisturizer, antioxidant, anti-hair loss, anti-dandruff, restoring the skin flora, acting on the hair cuticles, on the softness and silky appearance of hair, etc. These active ingredients can be obtained from plant materials, such as plant extracts or products of in-vitro plant culture or fermentation.

More specifically, the peptide(s) can be combined with at least one of the compounds chosen from the following compounds:
An anti-dandruff active agent acting as an antifungal agent: such as Zinc Pyrithione, Ketoconazole, Climbazole, Piroctone Olamine, Selenium disulphide or APISCALP™ (Sederma); and/or
An active agent which slows down or inhibits the development of the yeasts responsible for the dandruff state (of the Malassezia genus) and which acts favorably on the skin barrier, such as the Pal-KTTKS peptide (SEQ ID NO 1) or Pal-KTSKS (SEQ ID NO 2); and/or
A moisturizing active agent such as DuraQuench IQ™ (Croda) or the Shea Unsaponifiable™ (Sederma); and/or
An active agent rebalancing the skin microflora such as the active HAIRSPA™ (Sederma); and/or
A soothing active agent such as the active ingredient PACIFEEL™ (Sederma); and/or
An active agent for preventing hair loss and stimulating hair growth such as CAPIGENE™, CAPILECTINE™, PROCAPIL™ (Sederma); and/or
An active agent for strengthening the structure of damaged hair such as CERAMIDE A2™, CERAMIDE HO3™, HELIOGENOL™ (Sederma); and/or
An active agent for smoothing the hair such as FRUIT BIO™ (Sederma); and/or
An active agent for protecting hair and body hair from UV and IR radiation, such as VENUCEANE™.

The at least one peptide according to the invention can also be used in a vectorized form, by being bound, incorporated, or adsorbed on/to macro-, micro- or nanoparticles such as capsules, spheres, liposomes, oleosomes, chylomicrons, sponges, in the form of micro- or nano-emulsions, or adsorbed for example on powdery organic polymers, tales, bentonites, spores or exins and other inorganic or organic supports.

The hair care formulations can have a variety of forms, including hair conditioning, hair straightening formulations, straightening and curling products, perms, hair shampoos, pre-shampoo conditioners, aftercare shampoo, lotions, leave-on shampoos, styling products, leave-in hair products, waterless products, emulsions, 2 in 1 foaming emulsions, creams, masks, aerosol or non-aerosol foams, sprayable emulsions, products without emulsifier, aqueous or oily sprays, serums, gels, mild sulfate-free formulations, silicone-free formulations, products containing pigments, colored cosmetics, coloring formulations and shower products.

These hair care formulations comprising the at least one peptide according to the invention can comprise various other ingredients known to those skilled in the art such as, for example and without being exhaustive, cleaning agents, revitalizing agents for skin, styling agents, anti-dandruff agents, sequestering or complexing agents (EDTA and its salts), stabilizing agents, pearlescent and opacifying agents, plasticizers or coalescing agents, gelling agents, emollients, acidifying or alkalizing agents, hair growth promoters, anti-hair loss agents, perfumes, essential oils, polymers, proteins or derivatized proteins, silicones, sun protection compounds (filters), pigments, moisturizers, antioxidants, co-emulsifiers, film-forming agents, alpha-hydroxy acids, hair dyes, detergents, thickening agents, sheathing agents, texturizing agents, antiseptic agents, preservatives and surfactants.

These formulations are prepared according to methods known to those skilled in the art. The vehicle or carrier excipient for the formulation can be water, oil, or a powder, depending on the final destination of the formula: if, for example, it is a shampoo, it could be water; for other formulations, an oil will be preferred: such as, for example, vegetable oils (coconut, argan, jojoba, olive, sunflower, etc.), mineral oils, animal or synthetic oils. Synthetic esters, such as for example C12-15 Alkyl Benzoate, can also be used as an excipient. Liquid fatty alcohols, liquid fatty esters, solid fatty substances and in particular waxes, solid fatty esters and solid alcohols can also be used as support.

The present invention thus provides the use of at least one peptide according to the invention or of a composition comprising it, as defined above, for a non-therapeutic cosmetic treatment to improve the general condition of hair and treat their imperfections. The treatment consists of applying an effective amount of at least one peptide according to the invention to the hair in need thereof.

Preferably according to the invention, the treatment is topical.

The present invention covers a cosmetic, non-therapeutic topical treatment method for preserving and improving the appearance and general condition of the hair, in particular for preventing canities, comprising topical application to the hair of a subject in need thereof of an effective amount of a peptide or a mixture of peptides according to the invention or of a composition according to the invention comprising said peptide or mixture of peptides, the peptides being as defined above. The treatment aims all kind of hairs, including body hairs and head hair (including scalp hair, beard hairs, eyelashes and eyebrows).

By “non-therapeutic cosmetic treatment” is meant a treatment which is intended for hair in a healthy state (as opposed to a pathological state), with the aim of beautifying it or avoiding aesthetic disorders (as a preventive measure).

By “topical treatment” or “topical use” is meant an application which is intended to act where it is applied.

The peptide or the composition according to the invention can be applied locally to the targeted areas.

The “effective” amount depends on several factors, such as the person's age, condition, severity of the disorder, and how it is administered. An effective amount means a non-toxic amount sufficient to achieve the desired effect.

For example, a cosmetic hair treatment for the scalp of at least 4 weeks, preferably at least 12 weeks is recommended. This can be done in the form of a treatment at least 3 times a week, each time applying a dose of 5 ml of shampoo, then 5 ml of a leave-on lotion after shampooing, with in each product 1.5% of the active ingredient according to the invention dosed at 6000 ppm of peptide (according to the formulas of the Galenic part below).

According to other features, the cosmetic treatment method according to the invention can be combined with one or more other treatment methods targeting the hair, such as, for example, treatments by light therapy, by heat or by aromatherapy.

According to the invention, it is possible to provide devices with several compartments or kits intended for the implementation of the method described above, and which could comprise, by way of example, and without this being limiting, in a first compartment a composition containing the at least one peptide of the invention and in a second compartment an additional excipient and/or active, the compositions contained in said first and second compartments being considered here as a combination composition for simultaneous use, separate or spread in the time in particular in one of the treatments defined above.

According to a further subject matter, the present invention provides the use of at least one peptide according to the invention as an active ingredient in the preparation of a cosmetic composition for preserving and/or improving the general condition of the hair.

### DETAILED DESCRIPTION

The present invention will be better understood in the light of the following description of an embodiment, in-vitro evaluations, in-vivo evaluations, and hair formulations for carrying out the invention.

### A—Example of Peptide Synthesis According to the Invention: The Pal-PP-OH

The Pal-PP-OH peptide is prepared by peptide synthesis. An N-protected proline is attached to a resin via its terminal acid function. The amine function is deprotected and then the proline-resin is reacted with a proline derivative in the presence of a coupling agent (for example DCC (diclyclohexylcarbodiimide)/NHS (N-hydroxysuccinimide) or HBTU (2-(1H-benzotriazole-)1-yl)-1, 1, 3, 3-tetramethyluronium hexafluorophosphate)/HOBT (1-hydroxy-benzotriazole), then the same deprotection and coupling operation is repeated for palmitic acid. The peptide is then cleaved from the resin in an acidic medium and after precipitation, washing and drying, the product palmitoyl-prolyl-proline is obtained in solid form.

The peptides according to the invention can also be prepared by a biotechnological route, via a microorganism capable of producing it at least partially.

### B—Example of Preparation of a Composition According to the Invention Comprising the Dipeptide Pal-PP-OH (from Example A)

### Starting Materials:

The pure peptide, synthesized according to the synthetic method explained above;
Excipient: water, glycerin, 30% NaOH, orthophosphoric acid

Procedure: A selected quantity of peptide is dissolved in water at alkaline pH. Once the peptide is solubilized, the pH is lowered to 6 and the gycerin added.

In this specific example, 0.6% of Pal-PP peptide is mixed with the excipient to form an active ingredient used in dosage part D) below.

### C—In-Vitro Evalutations

The peptide according to the invention exhibits remarkable effects described below. The peptide prepared according to A) above is dissolved beforehand in DMSO, a solvent suitable for cell cultures. The solution of peptide in DMSO is then added to the culture medium. The final DMSO concentration in the media tested is 0.1%.

### 1—Anti-Aging Effect of the Peptide on Hair According to the Invention—Protective Against Oxidative Stress

To fight against the greying of hair, it is not only necessary to stimulate melanin production and pigment diffusion in the keratinocytes, but it is also advantageous to protect the melanocyte from the damage induced by age (intrinsic or chronological aging), premature aging linked to environmental factors (extrinsic aging), and by melanin production itself. Indeed, the melanin production generates oxygenated radical species (ROS), to which are added the effects of age, which also lead to the production of ROS. The major radical species is H₂O₂, which diffuses into the cell and can react with its essential components, which weakens the cell and reduces its production in quality and quantity. To control this, cells have an arsenal of defenses against oxidation, including catalase which detoxifies H₂O₂ to oxygen and water and glutathione, a small peptide which is also involved in the detoxification of H₂O₂ to H₂O via the GSH-peroxidase. The reduced form of glutathione (GSH) allows this enzyme to be recycled to restart its detoxification process.

In this antioxidant defense, the expression of the BCL-2 gene, encoding a protective protein of the cell against oxidative stress, is also decisive.

### 1.1—Reduction of the ROS Quantity

### Protocol:

Normal human melanocytes (NHM) almost at confluence are brought into contact with the peptide according to the invention for 24 hours. At the end of this contact, the cells are contacted with a DCFH-DA probe which is inserted into these cells. After rinsing to remove the excess probe, the cells are placed in contact with H₂O₂ to mimic oxidative stress. A case without oxidative stress is also realized. The reaction of H₂O₂ with the probe in the cell is measured by fluorescence. An estimate of the quantity of cells is carried out in parallel to harmonize the results.

### Results:

TABLE 4
Variation in the ROS quantity in NHM;
effect of the invention (n = 3):

| | Without H₂O₂ stress | |
|---|---|---|
| ROS quantity | (basal state) | After the H₂O₂ stress |
| Control | Reference | Reference |
| Pal-PP-OH at 10 ppm | −23%; p < 0.01 | −25%; p < 0.01 |
| Pal-PP-OH at 12.5 ppm | −24%; p < 0.01 | −33%; p < 0.01 |

### 1.2—Catalase Activity of the NHM

### Protocol:

NHM almost at confluence are brought into contact with the peptide according to the invention for 24 hours. At the end of this contact, the cells are crushed. The catalase activity extracted from these cells is measured using resorufin reagent. The latter combines with the H₂O₂ remaining in the reaction well after the action of cellular catalase. A protein assay using bicinchoninic acid (BCA) method is used to estimate the cell quantity to homogenize the obtained data.

### Results:

TABLE 5
Variation of the catalase activity in the NHM,
effect of the peptide of the invention (n = 3):

| Catalase activity | Variation (%); significance |
|---|---|
| Control | Reference |
| Pal-PP-OH at 10 ppm | +31%; p < 0.01 |
| Pal-PP-OH at 12.5 ppm | +37%; p < 0.01 |

### 1.3—Reduce Glutathion of the NHM

### Protocol:

NHM almost at confluence are brought into contact with the peptide according to the invention for 24 hours. After this contact, the cells are broken in an extraction buffer that preserves glutathione. The amount of GSH is determined by a specific fluorescent probe.

### Results:

TABLE 6
Variation in the amount of reduced glutathione in NHM, effect
of the peptide according to the invention (n = 6):

| Reduced glutathione concentration | Variation (%); significance |
|---|---|
| Control | Reference |
| Pal-PP-OH at 10 ppm | +28%; p < 0.05 |
| Pal-PP-OH at 12.5 ppm | +38%; p < 0.01 |

### 1.4—BCL-2 Marker of the NHM

### Protocol:

NHM are cultivated and brought into contact with the peptide according to the invention or its solvent. At the end of 72 hours of contact, the melanocytes are crushed, and the mRNA are recovered, converted into cDNA which are analyzed by a qRTPCR to evaluate the changes in the level of expression of the mRNA of BCL-2.

### Results:

TABLE 7
Variation in the expression of the BCL-2 gene in NHM, effect
of the peptide according to the invention (n = 4):

| | Variation (%); significance |
|---|---|
| Control | Reference |
| Pal-PP-OH at 10 ppm | +62%; p < 0.01 |
| Pal-PP-OH at 12.5 ppm | +99%; p < 0.01 |

These four sets of results show that the peptide according to the invention stimulates the antioxidant defenses of human melanocytes. The catalase enzyme is dose-dependently stimulated by +37% (p<0.01 vs. control). In parallel, the peptide of the invention stimulates the production of reduced glutathione (+38%, p<0.01 vs. control) and overexpressed the gene for the BCL-2 protein by +99% (p<0, 01 vs. control). Likewise, the peptide of the invention reduces the production of intracellular peroxide by 33% (p<0.01 vs. control) during oxidative stress.

### 2—Effect of the Peptide According to the Invention on Melanin Transfer

The production of melanin by tyrosinase in melanosomes is a first step. The latter must then be transferred from the melanocytes to the keratinocytes via the dendrites of the melanocyte. The size of the dendrites and the transfer capacity (phagocytosis) of pigmented melanosomes are also very important parameters for a good melanization.

### 2.1—at the Level of the Dendrites

### Protocol:

The cells are brought into contact with the peptide according to the invention for 48 hours. Then the melanocyte layers, once rinsed, are labeled using an antibody. The area occupied by the dendrites are obtain by image analysis on the photos taken, a parameter converted into the relative dendrite length/100 cells. At the same time, the cell nuclei are counterstained using Hoescht 33258 fluorescent dye to estimate the cell number and to harmonize the data.

### Results:

TABLE 8
Variation in the length of melanocyte dendrites, effect
of the peptide according to the invention (n = 3):

| Lenght of the dendrites | Variation (%); significance |
|---|---|
| Control | Reference |
| Pal-PP-OH at 5 ppm | +20%; p < 0.01 |
| Pal-PP-OH at 10 ppm | +33%; p < 0.01 |
| Pal-PP-OH at 15 ppm | +57%; p < 0.01 |

These results show the effect of the peptide according to the invention on the dendricity of NHM. The peptide stimulates the elongation of these extensions necessary for contact between melanocytes and keratinocytes.

### 2.2—at the Level of Melanosome Phagocytosis

### Protocol:

Normal human keratinocytes (NHK) are seeded and cultured until a mat of confluent cells is obtained. The cells are brought into contact with the peptide according to the invention for 24 hours and a solution of fluorescent microbeads mimicking the melanosomes of the melanocyte is added for 3 hours. The beads are phagocytosed (ingested) by the keratinocytes and quantified, after rinsing, by image analysis. In parallel, the cell nuclei are counterstained using the Hoescht33258 fluorescent dye to estimate the cell quantity and to harmonize the data.

### Results:

TABLE 9
variation in microbead phagocytosis by keratinocytes, effect
of the peptide according to the invention (n = 3):

| Number of microbeads per image analysis | Variation (%); significance |
|---|---|
| Control | Reference |
| Pal-PP-OH at 5 ppm | +50%; p < 0.05 |
| Pal-PP-OH at 10 ppm | +283%; p < 0.01 |

These results show the effect of the peptide according to the invention on the microbead phagocytosis stimulation which mimic melanosomes. This stimulation is dose-dependent and reaches+283% compared to the control (p<0.01).

With the dendricity data, it is therefore confirmed that the peptide according to the invention strongly promotes the transfer of melanosomes carrying melanin to the neighboring keratinocytes.

### 3—Effect of the Peptide According to the Invention on Melanogenesis

### 3.1—Melanin Production

### Protocol:

Normal human skin melanocytes (NHM), little or moderately melanin producing are used. These cells are cultured and brought into contact for 10 days with the peptide according to the invention at different concentrations or with its solvent in equivalent concentration (control). Culture media are changed every 2-3 days. After this contact, the cell mats are crushed, and the melanin is extracted from the cells. The melanin quantity is evaluated spectrophotometrically at 490 nm, using a standard range previously established from a melanin solution. Protein assay using the Bicinchoninic Acid (BCA) method is used to estimate the cell quantity to homogenize the obtained data.

### Results:

TABLE 10
variation in melanin production by moderately (§) and
poorly pigmented (§§) NHM after 10 days;
effect of the peptide according to the invention; n = 4:

| | Variation (%); | Variation (%); |
|---|---|---|
| Melanin concentration | significance (§) | significance (§§) |
| Control | Reference | Reference |
| Pal-PP-OH at 5 ppm | +48%; p < 0.01 | +4%; nsd |
| Pal-PP-OH at 10 ppm | +123%; p < 0.01 | +81%; p < 0.01 |
| Pal-PP-OH at 12.5 ppm | +223%; p < 0.01 | +113%; p < 0.01 |

nsd: non-significant data

These results show that the peptide according to the invention stimulates melanin production in the human melanocyte in culture, whether the cells originate from a moderately (§) or low-productivity (§ §) clone. This over-production is already clearly visible in photos taken before the crushing of the cell layers. The over-production is dose-dependent and very significant compared to what is observed in the negative control which remains very slightly pigmented even after 10 days.

### 3.2—Tyrosinase Activity

### Protocol:

The same culture and contact protocol as above are used with the melanocytes of two different donors with regard to their production of melanin (moderately and poorly pigmented).

After contact, the tyrosinase was extracted from the cells and its dopa-oxidase activity is assessed using the substrate L-DOPA at 37° C. The absorbance due to the production of Dopaquinone is measured at 490 nm and converted to activity units using a preset range. A protein assay using the Bicinchoninic Acid (BCA) method is used to estimate cell quantity and thus to homogenize the obtained data.

### Results:

TABLE 11
Variation in tyrosinase activity on moderately (§) and
poorly pigmented (§§) NHM after 10 days; effect
of the peptide according to the invention; n = 4:

| | Variation (%); | Variation (%); |
|---|---|---|
| Tyrosinase activity | significance (§) | significance (§§) |
| Control | Reference | Reference |
| Pal-PP-OH at 5 ppm | | +15%; p < 0.05 |
| Pal-PP-OH at 7 ppm | +14%; p < 0.01 | |
| Pal-PP-OH at 10 ppm | +27%; p < 0.01 | +74%; p < 0.01 |

These results show that the peptide according to the invention increases the tyrosinase activity in a dose-dependent manner, whether the cells originate from a low or moderately productive clone. The peptide can therefore promote a more active melanogenesis.

### 3.3—Effect of Genes Involved in Melanogenesis

### Protocol:

NHM are cultivated and brought into contact with the peptide according to the invention or its solvent. At the end of 72 h of contact, the melanocytes are crushed, and the mRNAs are recovered, converted into cDNA which are analyzed by qRTPCR, to estimate the changes in the expression level of the mRNAs. The expression of the following genes has been studied: MITF, TYRP1 and CREB.

### Results:

TABLE 12
variation in the expression of genes involved in melanogenesis,
effect of the peptide according to the invention; n = 4:

| | MITF | TYRP1 | CREB |
|---|---|---|---|
| Control | Reference | Reference | Reference |
| Pal-PP-OH at 10 ppm | +39%; p < 0.01 | +42%; p < 0.01 | +46%; p < 0.01 |
| Pal-PP-OH at 12.5 | +42%; p < 0.01 | +54%; p < 0.01 | +56%; p < 0.01 |
| ppm | | | |

These results show that the peptide according to the invention over-regulates dose-dependently the expression of several protein genes which are involved in melanogenesis: MITF, TYRP1 and CREB.

### 3.4—Effect on the Hair Follicule

### Protocol:

Follicles comprising their bulb with little pigment and isolated from a scalp plasty (resulting from cosmetic surgery—female donor, dark blond; 51 years old) are placed individually in the wells of a culture plate containing the culture medium and maintained for survival (37° C.; 5% CO₂).

Twelve follicles are reserved for T0 to serve as a reference for visual estimation and quantification of pigmentation but also for subsequent labeling of MC1R and MITF by immunohistology (six follicles for each marker).

Twelve follicles are placed in contact with the peptide according to the invention at 10 ppm in its solvent and twelve others in contact with the solvent alone for 8 days (0.1% DMSO in culture medium). At 8 days, all the follicles are stopped and fixed, then sectioned (7 μm) and marked with alunated carmine and photographed under a microscope. Sections of 6 of them (for each group) are used for visual and quantitative assessment by image analysis of their pigmentation (with specific filter to measure melanin). Sections of the other 6 are used for MC1R and MITF scoring, which is to be assessed by visual estimation.

### 3.4.1—Pigmentation Results:

TABLE 13
Variation in the melanin production in the follicle bulb after
8 days of contact with the peptide according to the invention
(n = 6 follicles/case; 22/23 photos/case in total):

| | Visual | Quantification by image analysis; |
|---|---|---|
| Melanin | assessment | % area occupied by black pixels |
| Control | + | Reference |
| Pal-PP-OH at 10 ppm | ++++++ | ×11.6; p < 0.01 |

The visual estimation indicates much stronger pigmentation in the 6 follicles that have been in contact with the peptide according to the invention for 8 days than in the 6 control follicles. Analysis of the photos indicates 11.6 times more dark pixels, precisely corresponding to the deposition of melanin, in the 6 follicles in contact with the peptide according to the invention. This difference is very significant.

### 3.4.2—Results for the MITF and MC1R Markers

After labeling the sections by immunohistology, the MC1R and MITF proteins appear dark. Compared to T0, the visual estimation indicates that MC1R is increased in the bulb of the cases treated with the peptide according to the invention while it remains stable in the control case. In parallel MC1R is increased more in the lower sheaths with the peptide according to the invention than in the control. MC1R is the a-MSH hormone receptor that induces melanization. The peptide according to the invention stimulated its production in the follicle.

Compared to T0, the visual estimation indicates that MITF is increased in the bulb of the cases treated with the peptide according to the invention while it decreases in the control case.

MITF is the protein that triggers the production of tyrosinase and TYRP1 and therefore melanin in melanosomes. The peptide according to the invention clearly induces the production of MITF, which causes the increase in melanin observed in cultured cells and in bulbs. This is in line with what is observed by qRT-PCR (see above), which showed increased expression of CREB, MITF and TRP-1 on melanocytes.

### D—Galenical

Different formulations are described below. Additional cosmetic active ingredients, possibly supporting and/or complementing the activity of the active ingredient according to the invention, can be added in the appropriate phase depending on their hydrophobic or hydrophilic nature. These ingredients can be of any category depending on their role(s), the place of application (hair of scalp, body hair, eyelashes, eyebrows, etc.), the desired final effect and the consumer. They are mentioned above in the description.

The formulas described below include an active ingredient based on a peptide according to the invention as described in point B above, containing 6000 ppm of peptide. These formulas are given as an indication with a recommended percentage of active ingredient at 1.5%. They could contain higher or lower percentages depending on the more or less pronounced effect sought.

### 1—Serum

TABLE 14

| Ingredient (INCI name) | Role | w/w % |
|---|---|---|
| Part A | | |
| Deionized water | — | qsp 100 |
| Carbomer | Rheology modifier | 0.25 |
| Part B | | |
| Potassium Sorbate | Preservative | qs |
| Part C | | |
| Deionized water | — | 2.00 |
| Sodium hydroxide 30% | pH adjuster | 0.20 |
| Part D | | |
| Alcohol | Solvent | 5.00 |
| Pentylen Glycol | Humectant | 3.00 |
| Arlasolve ™ DMI PC (Dimethyl | Humectant | 2.50 |
| Isosorbide) | | |
| Phenoxyethanol | Preservative | qs |
| Part E | | |
| Ingredient comprising the peptide of the | Active | 1.50 |
| invention | | |

Sprinkle the carbomer in the water and let swell for 30 minutes. Add part B to part A under stirring. Neutralize with part C to part A+B. Homogenize well by stirring. Pour part D into part A+B+C while stirring. Add part E and mix well.

A fluid, limpid and colorless gel is obtained, forming a serum of light non-greasy texture, which can be used to treat hair including body hair (application to the beard and to the torso for example).

### Examples of Additional Active Ingredients:

Neroli Floral Water™: active ingredient sold by Crodarom, bitter orange extract (Citrus aurantium amara) with soothing and regenerating properties.

### 2—Liquid Shampoo

TABLE 15

| Ingredient (INCI name) | Role | w/w % |
|---|---|---|
| Part A | | |
| Dezionised water | — | qs 100 |
| Potassium sorbate | Preservative | 0.10 |
| Citric acid | pH adjuster | 0.12 |
| Sodium citrate | pH adjuster | 1.20 |
| Part B | | |
| N-Hance ™ CCG 45 (Guar | Rheology | 2.00 |
| Hydroxypropyltrimonium Chloride) | modifier | |
| Part C | | |
| PEG-60 Hydrogenated Castor Oil | | 3.00 |
| Part D | | |
| Empicol ® ESB3 (Sodium Laureth Sulfate) | Surfactant | 20.00 |
| Crodateric ™ CAB 30 (Aqua (and) | Surfactant | 5.00 |
| Cocamidopropyl Betaine) | | |
| Phenoxyethanol | Preservative | 0.80 |
| Crothix ™ Liquid (PEG-150 Pentaerythrityl | Surfactant | 3.00 |
| Tetrastearate (and) Aqua (and) PEG-6 Caprylic/ | thickener | |
| Capric Glycerides) | | |
| Part E | | |
| Deionized water | — | 1.00 |
| Lactic acid | pH adjuster | 0.05 |
| Part F | | |
| Ingredient comprising the peptide of the invention | Active | 1.50 |

Weigh part A well. Sprinkle part B into part A, stirring normally and mix well for 1 hour. Heat part A+B to 55° C. in a water bath, mix well. Weigh and heat part C to 55° C. in a water bath. Add Part C to Part A+B under rapid stirring. Add the ingredients from part D, one at a time, to part A+B, stirring normally. Adjust the pH with part E to pH=5.90+/−0.10. Add part F, mix well.

A white opaque viscous gel is obtained.

### Examples of Additional Active Ingredients:

APISCALP™: active ingredient sold by Sederma, based on a CO₂ supercritical extract of Apium graveolens seeds, for the treatment of irritated scalp, which helps alleviate dandruff and relieves itchy scalp (added in the part C).

Zinc Pyrithione: anti-dandruff agent (added to part A).

### 3—Rince Oil

TABLE 16

| Ingredient (INCI name) | Role | w/w % |
|---|---|---|
| Part A | | |
| Crodamol ™ OP (INCI name: Ethylhexyl | Emollient | qsp 100 |
| Palmitate) | | |
| Crodamol ™ AB (C12-15 Alkyl Benzoate) | Light touch | 10.00 |
| | emollient | |
| Crodamol ™ STS (PPG-3 Benzyl Ether | Emollient with | 5.00 |
| Myristate) | shiny effect | |
| Crodamol ™ IPIS (Isopropyl Isostearate) | Emollient to | 5.00 |
| | aid in rinsing | |
| Seatons Coconut Oil (Cocos Nucifera | Natural oil | 5.00 |
| (Coconut) Oil) | | |
| Phenoxyethanol | Preservative | 0.80 |
| Dl Alpha Tocopherol | Antioxidant | 0.20 |
| Part B | | |
| Cithrol ™ 10GTIS (PEG-20 Glyceryl | Cleansing | 15.00 |
| Triisostearate) | surfactant | |
| Pentylene Glycol | Humectant | 3.00 |
| Arlasolve ™ DMI PC (Dimethyl Isosorbide)1 | Solubilizer | 2.50 |
| Ingredient comprising the peptide of the | Active | 1.50 |
| invention | | |

Weigh and mix part A. Weigh and mix part B. Add part B to part A under normal stirring. A liquid and clear yellow oil is obtained, preferably to leave on, then rinsed, which can be used both to treat hair including body hairs (for example as massage oil on the torso).

### Examples of Additional Active Ingredients:

Crodabond™ CSA: active ingredient sold by Croda, a blend of hydrogenated castor oil and sebacic acid copolymer, which seals raised cuticles (the outermost layer of the hair shaft) and repairs split ends (added to part B).

Phytolea™ Baobab EC: active ingredient sold by Crodarom, Adansonia digitata seed oil, regenerating and antioxidant due to its fatty acids, and vitamin E and A content (added to part A).

### 4—Solid Shampoo, for Example in the Form of a Stick

TABLE 17

| Ingredient (INCI name) | Role | w/w % |
|---|---|---|
| Part A | | |
| Crodasinic ™ LS30 (Aqua (and) Sodium | Surfactant | 20.00 |
| Lauroyl Sarcosinate) | | |
| Phytofoam ™ (Aqua (and) Acacia Concinna | Surfactant | 5.00 |
| Fruit Extract (and) Balanites Aegyptiaca Fruit | | |
| Extract (and) Gypsophila Paniculata Root | | |
| Extracts) | | |
| Pentylen Glycol | Humectant | 3.00 |
| Arlasolve ™ DMI PC (Dimethyl Isosorbide) | Humectant | 2.50 |
| Unicert Yellow 08005-J solution 0.5% (Aqua | Coloring | 0.70 |
| (and) CI 19140) | agent | |
| Unicert Red K7057-J solution 0.5% (Aqua | Coloring | 0.30 |
| (and) CI 17200) | agent | |
| Part B | | |
| Crodacol ™ CS90 (Cetearyl Alcohol) | Wax, cohesion | 19.00 |
| | agent | |
| Crodazosoft ™ BDQ (Quaternium-91 (and) | Conditioning | 2.00 |
| Cetrimonium Methosulfate (and) Cetearyl | surfactant | |
| Alcohol) | | |
| Syncrowax ™ HRC (Tribehenin) | Thickener | 2.00 |
| Part C | | |
| Ingredient comprising the peptide of the | Active | 1.50 |
| invention | | |
| Part D | | |
| Kaolin | Excipient | qsp 100 |

Heat part A to 75° C. in a water bath. Heat part B to 75° C. in a water bath. Add part C to part A and mix well, while stirring pale normally. Add part B to part A+C with light rapid stirring, still in a water bath. Add part D to the previous part, mix well. Pour into molds immediately.

An opaque solid shampoo is obtained.

### Examples of Additional Active Ingredients:

Matcha Tea Extract™: antioxidant and purifying active ingredient sold by Crodarom based on Camellia Sinensis leaf extract, (added to part C.

Hairspa™: active ingredient sold by Sederma, soothing and moisturizing for the scalp, based on lactitol and xylitol, (added to part C).

### 5—Energizing Effect Wax

TABLE 18

| Ingredient (INCI name) | Role | w/w % |
|---|---|---|
| Part A | | |
| Crodamol ™ AB (C12-15 Alkyl | Light touch | qs 100 |
| Benzoate) | emollient | |
| Oleocraft ™ LP-20 (Polyamide-8) | Oily stucturing | 17.00 |
| | polymer | |
| Crodamol ™ IPIS (Isopropyl Isostearate) | Light touch | 5.00 |
| | emollient | |
| Crodamol ™ ML (Myristyl Lactate) | Emollient | 4.00 |
| Span ™ 120 (Sorbitan Isostearate) | Emulsifier | 2.00 |
| Crodamol ™ STS (PPG-3 Benzyl Ether | Shining effect | 1.00 |
| Myristate) | emollient | |
| Crodazosoft ™ BDQ (Quaternium-91 (and) | Conditioning | 1.00 |
| Cetrimonium Methosulfate (and) Cetearyl | surfactant | |
| Alcohol) | | |
| Part B | | |
| Pentylen Glycol | Humectant | 3.00 |
| Arlasolve ™ DMI PC (Dimethyl Isosorbide) | Solubilizer | 2.50 |
| Phenoxyethanol | Preservative | 0.40 |
| Part C | | |
| Cithrol ™ 10GTIS (PEG-20 Glyceryl | Emulsifier | 10.00 |
| Triisostearate) | | |
| Ingredient comprising the peptide of the | Active | 1.50 |
| invention | | |

Heat part A to 80° C. in a water bath. Mix well until melted and perfectly homogenized. Add part B to part A and mix well at 55° C. Pour part C into the previous part and mix. Pour hot immediately into a conditioning pot.

A clear gel is obtained which can be used on the hair and body hair.

### Examples of Additional Active Ingredients:

NG Shea Unnsaponifiables™: (Butyrospermum Parkii (Shea) Butter (and) Butyrospermum Parkii (Shea) Butter Unsaponifiables), active ingredient sold by Sederma, improves hydration (added to part A).

Phytole™ Cranberry EC: active scalp moisturizer sold by Crodarom (Vaccinium Macrocarpon (Cranberry) Seed Oil) (added to end of part A).

### 6—Regenerating and Nourishing Mask

TABLE 19

| Ingredient (INCI name) | Role | w/w % |
|---|---|---|
| Part A | | |
| Deionized water (Aqua) | — | qs 100 |
| Potassium sorbate | Preservative | 0.10 |
| Part B | | |
| Crodacol ™ CS90 (Cetearyl Alcohol) | Emulsion | 4.50 |
| | stabilizer | |
| Crodamol ™ STS (PPG-3 Benzyl Ether | Shining effect | 4.00 |
| Myristate) | emollient | |
| Cutissential ™ Behenyl 18 MEA | Cationinc | 3.00 |
| (Behentrimonium Methosulfate (and) C10-40 | surfactants | |
| Isoalkylamidopropylethyldimonium Ethosulfate | | |
| (and) Cetyl alcohol) | | |
| Crodazosoft ™ BDQ (Quaternium-91 (and) | Conditioning | 2.70 |
| Cetrimonium Methosulfate (and) Cetearyl | surfactant | |
| Alcohol) | | |
| Part C | | |
| Pentylen Glycol | Humectant | 3.00 |
| Arlasolve ™ DMI PC (Dimethyl Isosorbide) | Solubilizer | 2.50 |
| Phenoxyethanol | Preservative | 0.80 |
| Part D | | |
| Unicert Yellow 08005-J solution 0.1% (Aqua | Coloring agent | 0.15 |
| (and) CI 19140) | | |
| Part E | | |
| Deionized water (Aqua) | — | 1.50 |
| Sodium Hydroxide 30% | pH adjuster | 0.15 |
| Part F | | |
| Ingredient comprising the peptide of the | Active | 1.50 |
| invention | | |

Heat part A to 85° C. in a water bath. Heat part B to 90° C. in a water bath. Weigh part C and mix well. Pour part C into part A, stirring normally. Disperse part B in part A+C with vigorous stirring, homogenize well. Successively add parts D to F, homogenizing well.

Cutissential™ Behenyl 18 MEA: excipient sold by Croda used in the formula to replenish the lipid layer of the cuticles for a healthier appearance of hair and body hair and to promote excellent wet detangling performance.

A yellow opaque viscous emulsion is obtained which can be used for the hair and body hair.

### Examples of Additional Active Ingredients:

Procapil™: active ingredient sold by Sederma, preventing hair loss, comprising a blend of apigenin, oleanolic acid and Biot-GHK peptide (added to part D).

Crodarom Manuka Honey™: active ingredient sold by Crodarom based on a honey extract, helps repairing damaged hair (added to part D).

Ceramide HO3: active ingredient sold by Sederma, helps repairing damaged hair and promotes hydration (added to part B).

### 7—Leave-on Protective Spray

TABLE 20

| Ingredient (INCI name) | Role | w/w % |
|---|---|---|
| Part A | | |
| Deionized water (Aqua) | — | qs 100 |
| Potassium sorbate | Preservative | 0.10 |
| Viscaress ™ HPD (Polyquaternium-37 (and) | Conditioning | 1.50 |
| Hydrogenated Polydecene (and) Trideceth-6 | polymer | |
| (and) Aqua) | | |
| Lustreplex ™ (Polyquaternium-70 (and) | Cationic | 1.50 |
| Dipropylene Glycol) | surfactant | |
| Part B | | |
| Alcohol | Drying effect | 5.00 |
| Pentylen Glycol | Humectant | 3.00 |
| Arlasolve ™ DMI PC (Dimethyl Isosorbide) | Solubilizer | 2.50 |
| Crodamol ™ STS (PPG-3 Benzyl Ether | Shining effect | 1.00 |
| Myristate) | emollient | |
| Tween ™ 20 (Polysorbate 20) | Solubilizer | 0.90 |
| Phenoxyethanol | Preservative | 0.80 |
| Part C | | |
| Deionized (Aqua) | — | 1.00 |
| Sodium hydroxyde 30% | pH adjuster | 0.10 |
| Part D | | |
| Voluminis ™ (Ethyltrimonium Chloride | Hair | 1.00 |
| Methacrylate/Hydrolyzed Wheat Protein | conditioner | |
| copolymer) | | |
| Part E | | |
| Ingredient comprising the peptide of the | Active | 1.50 |
| invention | | |

Pour part B into part A with normal stirring. Adjust the pH with part C to 5.80+/−0.20. Add part D to part A+C under stirring. Add part E to the previous part and mix.

An opaque fluid emulsion is obtained.

### Examples of Additional Active Ingredients:

Phytessence™ Hazel Leaf: active ingredient sold by Crodarom based on a leaf extract of Corylus avellana, restoring vitality and tone to the scalp (added to Part D).

Venuceane™: active ingredient sold by Sederma based on a fermentation extract of Thermus termophillus Ferment, preventing damage from UV and IR radiations (added to part D).

### E—In-Vivo Evaluations

### 1—Generalities

The in-vivo tests were carried out by applying the hair lotion described above in point 1) in the Galenic part. This lotion comprises 90 ppm of the peptide of the invention. Four independent studies were conducted with a total of 84 volunteers (average age of the entire panel of selected volunteers was 42 years). All four studies were conducted over a wide range of geographical locations, times, seasons, application sites, phototypes and methods. The details are specified in the below table.

### Comparative Protocols of the Tests Carried Out:

TABLE 21

| Test | | | | |
|---|---|---|---|---|
| geographical site | Site 1 | Site 2 | Site 3 | Site 4 |
| Numbers of | N = 21 | N = 19 (N = 18 at | N = 25, (men) | N = 17, (13 |
| volunteers (N), | (Men), | 4 months), | | men/4 women) |
| gender | (N = 7 at 6 | (15 men/4 women) | | |
| | months: | (N = 10 at 8 months: | | |
| | persistence of | persistence of the | | |
| | the effect | effect beyond | | |
| | beyond | testing) | | |
| | testing) | | | |
| Mean age | 47 years | 41 years | 42 years | 38.4 ans |
| Extremes | [35-61] | [31-45] | [33-57] | [28-51] |
| Phototypes | II-III | III-IV | II-III | II-III |
| Applications | 5 ml/d/3 | 5 ml/d/3 months | 5 ml/d/3 | 5 ml/d/3 |
| | months | (N = 19) and | months (N = 25) | months (N = 17) |
| | (N = 21) + | 4 months (N = 18) + | | |
| | 3 months | 4 months without | | |
| | without | product (N = 10) | | |
| | product (N = 7) | | | |
| Seasons | Autumn- | Summer-Autumn- | Summer | Autumn |
| | Winter | Winter | | |
| Measure location | Behind - side | Top - side of the | Side of the head | Top - side of the |
| | of the head | head | | head |
| Measure method | Photos; | Photos; | Photos; | Photos; |
| | Image analysis | Image analysis by | Image analysis | Image analysis |
| | by Image J | Image J | by Image J | by Image J |
| | | Microphotos | | |
| Photographic | Headscan ™ | Headscan ™ | Headscan ™ | Visia-CR ™ |
| equipment | photographic | photographic bench | photographic | System |
| | bench | Polarized/crossed | bench | Polarized/ |
| | Polarized/ | mode | Polarized/ | crossed mode |
| | crossed mode | | crossed mode | |

| | |
|---|---|
| Statistics | Student t or Wilcoxon Test; bilateral tests, paired series. Final effect at final |
| | T vs. T0 |

These four studies were not done against placebo. A greying study was previously done on a placebo formula. Thirty volunteers were included (average 51 years [33-62 years]; 21 men, 9 women) with an application of lotions for 3 months at least 3 times a week. No significant greying reduction effect was observed or measured in this study on either the beard or the temples. This confirms that the application of a placebo lotion, even with daily massage, has no effect on greying.

### 2—Details of Methods

### 2.1—Standardized Photo Taking

All the studies used a complex photographic system, with the camera mounted on a bench to ensure perfect repositioning of the volunteer's head at all study times. For each volunteer, profile photos, and in some studies also of the back or the top of the head, were taken in cross-polarized mode to eliminate any parasitic shine so as not to interfere with the image analysis photo treatment. These photos are used to both get an overview of the grey hair intensity and, by their quality, to work on a smaller isolated area.

### 2.2—Treatment System of the Photos

The evaluation of the area occupied by grey/white hair was made in the photos with the NIH-USA™ Image J software by targeting the areas of interest.

Commonly, a short hair size (2 cm for example) was requested, except for test site 2, and that this size be the same for a volunteer at different measurement times to facilitate before-after comparisons.

Also, in common, each original photo, in color, was converted to an image with grey levels, the same level filter (thresholding operation) was applied before and after to the photos of the same volunteer. Non-black hair where thus selected. At the end, a binarization operation (black/white) was carried out to separate the non-black hairs (grey and white) from the blacks and to quantify the former.

### 2.3—Standardized Micro-Photos Using TrichoScan®

Only one study (at site 2) used this technology (a new tool for analyzing hair growth) which has the advantage of providing data on a very small area and seeing the hair individually. The scalp area is identified and shaved a few days before the photo, and this at different times. Widely used for the study of hair loss, with this technology, thanks to its high magnification, it is also possible to work on hair coloring. The TrichoScan® system includes an epiluminescence microcamera system.

2.4—Evaluation of the Number of White Hairs and Non-White Hairs The TrichoScan® software was used to determine the number of white hairs, the number of black hairs and the calculation of the black/white ratio. This ratio increases with the number of dark hairs when the treatment acts.

### 3—Results

No adverse effects were reported for these volunteers whether at 3 or 4 months. There were no negative feedbacks neither 3 months after the last application. This, together with the tolerance tests, shows the safety of the peptide of the invention on large groups and over a long period of time.

### 3.1—Grey Hair Density at 3 Months, Comparison of Effects in 3 Studies

Table 22 compares the quantitative effects obtained by 3 of the 4 groups on a common area, at the same application time (3 months) and a common parameter, the density of grey/white hair (although measured differently according to each group).

TABLE 22
variation in grey/white hair density measured on one side (profile), effect of the
peptide according to the invention after 3 months of application; N = 3 studies

| | | | | | | | Mean |
|---|---|---|---|---|---|---|---|
| Grey hair | 21 volunteers | | 25 volunteers | | 17 volunteers | | T 3 m |
| density | T0 | T 3 m | T0 | T 3 m | T0 | T 3 m | vs. T0 |
| Mean (%) | 7.65 | 4.78 | 3.10 | 1.90 | 9.50 | 7.50 | |
| +/−SD | +/−5.52 | +/−4.29 | +/−3.00 | +/−1.80 | +/−5.40 | +/−5.20 | |
| Variation vs. T0 | −37.5% | | −38.7% | | −21.1% | | −32.43% |
| Significance | p < 0.01 | | p < 0.01 | | p < 0.05 | | 81% |
| Maximum; | −70%; 100% | | −86%, 68% | | −67%; 76% | | |
| respondents | | | | | | | |

After 3 months of application, the grey hair density decreased on the temples by 32.4% on average (3 tests) with extremes between 21.1% and 38.7%. These decreases are very significant (p<0.01) and concern almost all the volunteers (68 to 100% according to the study).

### 3.2—Comparison Between Two Application Areas

In one of the studies, the measurements were taken on the side of the face and behind the head on the same volunteers, allowing to compare the peptide effects on two areas for the same volunteer group.

TABLE 23
Variation in grey/white hair density measured on a temple
and behind the head, effect of the peptide according to
the invention after 3 months of application; N = 1 study

| | 21 volunteers (temple) | | 21 volunteers (behind the head) | |
|---|---|---|---|---|
| Grey hair density | T0 | T 3 m | T0 | T 3 m |
| Mean (%) | 7.65 | 4.78 | 4.82 | 3.28 |
| +/−SD | +/−5.52 | +/−4.29 | +/−3.60 | +/−2.67 |

| | | |
|---|---|---|
| Variation vs. T0 | −37.5% | −31.9% |
| Significance | p < 0.01 | p < 0.01 |
| Maximum; respondents | −70%; 100% | −70%; 95% |

After 3 months of application, the grey hair density decreased on the temples and on the back of the head by 37.5% and 31.9% respectively. These reductions are very significant (p<0.01) and concern almost all the volunteers (100 and 95% depending on the area).

### 3.3—Comparison of the Effect Between 3 and 4 Months

One of the tests (site 2), conducted on the top of the head, was conducted for a total of 4 months with a point at 3 months to assess the effect over time. Table 24 compares the effect obtained at T 3 months and T 4 months.

TABLE 24
Variation in the grey/white hair density measured on the
top of the head, effect of the peptide according to the
invention after 3 and 4 months of application; N = 1 study

| Grey hair | 19 volunteers | | 18 volunteers | |
|---|---|---|---|---|
| density | T0 | T3 m | T0 | T 4 m |
| Mean (%) | 44.12 | 40.22 | 44.65 | 39.78 |
| +/−SD | +/−15.19 | +/−12.72 | +/−15.45 | +/−14.03 |
| Variation vs. | −8.80% | | −10.90% | |
| T0 | | | | |

| | | |
|---|---|---|
| Significance | p < 0.01 | p < 0.01 |
| Maximum | −26% | −23% |
| Respondents | 79% | 84% |

The study shows that after 4 months of use, the decrease in grey/white hair observed on the top of the head increases further compared to that observed at T 3 months (10.9% vs. 8.8%, both being very significant: p<0.01). The percentage of volunteers with improvement also increases slightly (79% to 84%).

### 3.4—Test by TrichoScan®

TABLE 25
Variation in the non-white/white hair ratio measured on the
temples using the Trichoscan ®, effect of the peptide
of the invention after 4 months of application; N = 1 study

T 4 months
18 volunteers

| Ratio | T0 | T 4 m |
|---|---|---|
| Mean | 2.01 | 2.56 |
| SD | +/−1.10 | +/−1.07 |

| | |
|---|---|
| % Variation vs. T0 | +27.4% |
| Significance | p < 0.05 |
| Maximum (variation); respondents | +385%; 72% |

An increase in the black/white hair ratio of approximately +27.4% at T 4 months (p<0.05) is measured. Therefore, this technique, which is very different from that using image analysis because it uses a microscope-type probe, confirms the reduction in the number of white hairs following the use of the peptide according to the invention.

3.5—Persistence of the Effect 3 or 4 Months after the End of the Tests

For two different tests, the persistence of the effect of the peptide according to the invention was evaluated over time. For this, after 3 or 4 months of application, all the volunteers stopped using the peptide according to the invention.

In the first test with 4 months of application, the persistence of the effect without application was evaluated in 10 volunteers 4 months later. The measurement was taken on the top of the head.

In the second test with 3 months of application, the persistence of the effect without application was evaluated in 7 volunteers 3 months later. The measurement was taken behind the head.

TABLE 26
Variation in the grey/white hair density measured behind the head (N =
7) or on the top of the head (N = 10) - effect of the peptide according
to the invention at 3 or 4 months after the last application; N = 2 studies

| Grey hair | 10 volunteers | | | 7 volunteers | | |
|---|---|---|---|---|---|---|
| density | T0 | T 4 m | T 8 m | T0 | T 3 m | T 6 m |
| Moyenne (%) | 46.32 | 39.36 | 36.04 | 4.99 | 3.14 | 3.00 |
| +/−SD | +/−17.30 | +/−14.97 | +/−16.81 | +/−3.09 | +/−2.60 | +/−2.26 |
| Variation vs. T0 | | −15.04% | −22.20% | | −37.2% | −39.9% |
| Significance | | p < 0.01 | p < 0.05 | | p < 0.05 | p < 0.01 |
| Maximum | | −22%; 100% | −45%; 80% | | −70%; 100% | −60%; 100% |
| Repondents | | | | | | |

Two independent studies, involving a smaller number of volunteers, show, after 3 or 4 months of daily use, that the decrease in grey hair is still very clear 3 or 4 months after the last application. The regression values are even slightly increasing (−22.20% against −15.04%, and −39.9% against −37.2%). It shows that the peptide according to the invention at 90 ppm re-pigment the grey hair, and this effect is advantageously maintained in the months following the end of the application.

### 4—In-Vivo Tests Conclusion

The peptide of the invention at 90 ppm in a hair lotion allowed a clear hair re-pigmentation of several dozen people included in four independent tests. Re-pigmentation is not dependent on the application site. Clear effects have been observed on the temples, above or behind the head. This effect does not depend on the test geographical location, nor on the quantification methods used since the four studies, carried out independently of one another, all showed a net re-pigmentation effect in almost all the volunteers. The women presented a re-pigmentation like the men. The phenomenon of persistence of the effect three months after the end of the test is particularly interesting because it reflects the fact that the peptide of the invention exhibits a “delay” effect, that is to say an effect that is prolonged over time, beyond the test, without the need to apply again the peptide. It shows that advantageously the peptide used according to the invention acts by reactivating one of the mechanisms leading to pigmentation that was altered over time and over the age of the subject.

1. Use of at least one peptide of Formula 1: X-(Xaa)n-P*P*-(Xaa)ₘ-Z
wherein:
P* corresponds to a Proline, analog or derivative thereof;
Xaa is an amino-acid selected from P*, Glycine, Alanine, Valine, Leucine, Isoleucine, Arginine and Phenylalanine, analogs and derivatives, the Xaa being selected independently from each others;
n and m are integers selected indepently from each others equal to 0, 1, 2, 3 or 4, with n+m≤8; and
at the N-terminal end, X is selected from H, —CO—R¹, —SO₂—R¹ or a biotinoyle group;
at the C-terminal end, Z is selected from OH, OR¹, NH₂, NHR¹ or NR¹R²;
R¹ and R² are, independently from each other, selected from an alkyl, aryl, aralkyl, alkylaryl, alkoxy, saccharide and aryloxy groups, that can be linear or branched, cyclic, polycyclic, unsaturated, hydroxylated, carbonylated, phosphorylated and/or sulfured, said group having 1 to 24 carbon atoms and optionally having in its skeleton one or more O, S and/or N heteroatoms atoms;
for a cosmetic non-therapeutical hair treatment adapted to prevent hair depigmentation.

2. Use according to claim 1, wherein the treatment is an antioxidizing treatment of the melanocytes present in the hair follicule.

3. Use according to claim 1, wherein the treatment is for promoting the transfer of melanin to the keratinocytes by the dendrites.

4. Use according to claim 1, wherein the treatment is further adapted for hair repigmentation.

5. Use according to claim 4, wherein the treatment is adapted for a long-lasting hair repigmenation.

6. Use according to claim 1, wherein the treatment is topical.

7. Use according to claim 1, wherein P* is a Proline.

8. Use according to claim 1, wherein n and m are independently from each other equal to 0, 1, or 2.

9. Use according to claim 1, wherein the peptide is modified at the N-terminal end and/or at the C-terminal end.

10. Use according to claim 1, wherein R¹ and/or R² is an alkyl chain having 1 to 24 carbon atoms.

11. Use according to claim 1, wherein the group CO—R¹ is selected from an octanoyl (C8), decanoyl (C10), lauroyl (C12), myristoyl (C14), palmitoyl (C16), stearoyl (C18), biotinoyle, elaydoyl, oleoyle and lipoyl group.

12. Use according to claim 1, wherein X is an acyle group CO—R¹ and Z is selected from OH, OMe, OEt and NH₂.

13. Use according to claim 1, wherein the peptide is the Pal-PP-OH.

14. Use according to claim 1, wherein the peptide is used in a vectorized form, being bound, incorporated, or adsorbed on/to macro-, micro-, or nano-particles such as capsules, spheres, liposomes, oleosomes, chylomicrons, sponges, as micro- or nano-emulsions, or adsorbed for example on powdery organic polymers, talcs, bentonites, spores or exins and other inorganic or organic supports.
